(19) United States
(12) Patent Application Publication
(10) Pub. No.: US 2018/0273790 A1
(43) Pub. Date: Sep. 27, 2018

(54) METHOD OF MANUFACTURING POLYMER COATED FERROMAGNETIC PARTICLES

(71) Applicants: TOKYO INSTITUTE OF TECHNOLOGY, Meguro-ku (JP); TAMAGAWA SEIKI KABUSHIKI KAISHA, Iida-shi (JP)

(72) Inventors: Hiroshi HANDA, Yokohama-shi (JP); Kosuke NISHIO, Yokohama-shi (JP); Nobuyuki GOKON, Yokohama-shi (JP); Masanori ABE, Meguro-ku (JP)

(73) Assignees: TOKYO INSTITUTE OF TECHNOLOGY, Meguro-ku (JP); TAMAGAWA SEIKI KABUSHIKI KAISHA, Iida-shi (JP)

(21) Appl. No.: 15/991,728

(22) Filed: May 29, 2018

Related U.S. Application Data
(63) Continuation of application No. 15/612,760, filed on Jun. 2, 2017, now abandoned.
Application No. 15/612,760 is a continuation of application No. 14/831,381, filed on Aug. 20, 2015, now abandoned.
Application No. 14/831,381 is a continuation of application No. 12/294,770, filed on Nov. 28, 2008, now abandoned.

(51) Int. Cl. C09D 125/14; G01N 33/543; C08F 292/00; C08F 2/24; C08F 2/44; C08F 212/08; C08F 212/36; C08F 220/32; C08K 3/22

(52) U.S. Cl. CPC C09D 125/14; G01N 33/5434; C08F 292/00; C08F 2/24; C08F 2/44; C08K 2003/2275; C08F 212/36; Y10T 428/2998; C08F 2220/325; C08K 3/22; C08F 212/08

(57) ABSTRACT

A method of manufacturing a polymer coated single ferromagnetic particle is provided. The method includes hydrophobizing one single hydrophilic ferromagnetic particle by absorbing an aliphatic acid having a hydrophobic aliphatic group and a hydrophilic acid group onto the single ferromagnetic particle to obtain a single hydrophobic ferromagnetic particle; emulsifying the one single hydrophobic ferromagnetic particle with a monomer liquid comprising a nonionic surface activating agent which re-hydrophilizes the one single hydrophobic ferromagnetic particle, to obtain an emulsified liquid; adding a radical addition initiator to the emulsified liquid ; and emulsion polymerizing the monomer by radical addition polymerization.

### CROSS REFERENCE TO RELATED APPLICATIONS

This application is a continuation application of U.S. application Ser. No. 15/612,760, filed Jun. 2, 2017, the disclosure of which is incorporated herein by reference in its entirety. U.S. Application No. 15/612,760 is a continuation application of U.S. application Ser. No. 14/831,381, filed Aug. 20, 2015, now abandoned, the disclosure of which is incorporated herein by reference in its entirety. U.S. application Ser. No. 14/831,381 is a continuation of U.S. application Ser. No. 12/294,770, filed Nov. 28, 2008, now abandoned, the disclosure of which is incorporated herein by reference in its entirety. U.S. application Ser. No. 12/294,770 is the National Stage of PCT/JP06/306197, filed Mar. 27, 2006, the disclosure of which is incorporated herein by reference in its entirety.

### TECHNICAL FIELD

The present invention relates to a method of manufacturing ferromagnetic particles having excellent polymer coating showing good response sensitively to magnetic field, to the polymer coated ferromagnetic particles, and to usage of the polymer coated ferromagnetic particles.

### BACKGROUND ART

Materials showing ferromagnetic characteristics in bulk state such as ferrite materials do not show ferromagnetic characteristics but show super-paramagnetic behavior when the particle size is very small. Furthermore, when the particle size is equal to or larger than a critical size, the materials show ferromagnetic behavior and apt to form particle aggregates due to the magnetic attractive force between particles. When aggregates are formed, the particles behave like particles with effectively larger particle size, and are prone to yield particle dispersion instability in liquid. Then various inconveniences for applying the particles occur. There are many efforts of using the particles to applications avoiding aggregate formation by weakening the cohesive force between the ferromagnetic particles applying various methods. The methods of coating the ferromagnetic particles with polymer to provide steric hindrance to cope with the attractive forces between the ferromagnetic particles, and methods of providing electrostatic repulsive force between functional groups are examples of the methods. The polymer coated ferromagnetic particles are widely used in many industrial fields, for example as magnetic carrier of the toner for electro-photography, magnetic colloid, magnetic ink, and magnetic paint can be cited. Furthermore, the particles are widely used as raw materials of molded products, for example, as raw particles for molded plastic magnets.

However, problems are pointed out to the existing polymer coated ferromagnetic particles of relatively large particle sizes of micron meter order with large particle size scattering. There were little or no methods for controlling polymer coating thickness and shape, especially for particles having average diameter of 500 nm or less. Therefore, a new technology that can attain the control has been desired. In biological science and medical activity in recent years, polymer coated magnetic particles such as polymer coated ferrite particles, are used as a carrier of a bio-separation, and a magnetic marker for bio-molecules. In these applications also, a new method of obtaining thickness and shape controlled coated particles having average diameter of 500 nm or less with good particle diameter uniformity has been desired.

Inventors of the present invention developed bonded composite fine particles of polymer particles and ferrite fine particles (Patent reference 1: JP-A 2002-090366 (KOKAI)), and also developed ferrite particles bonded with organic material in which the organic material are directly bonded to the ferrite particles (Patent reference 2: WO 03/066644 A1), and have shown there that these have desirable characteristics in usage of purification and identification for the specific drug substances.

A research work related to the work is found for example in “Scientific and Clinical Applications of Magnetic Carriers, Plenum Press 1997” (Non-patent reference 1), in which polymer fine particles bonded to ferrite having magnetic response and their application are described.

A patent publication for coating inorganic fine particles such as ferrite particles with polymer and introducing functional groups that react with biological molecules or chemical molecules on the polymer coats and applying the particles for detecting, separating and refining specified bio-molecules using the specific interaction characteristics of the molecules is found in JP-A 2003-513093 (KOKAI) (Patent reference 3).

When ferrite particles are applied as ferromagnetic particles of polymer coated ferromagnetic particles, the particle diameter is desired to be at an extent of 100 nm or less than 100 nm for sustaining colloidal dispersed state in liquid. The particles, on the other hand, are hoped to have magnetization sufficient to respond to handling using magnetic field.

For this purpose, the particle size is required not to be too small such that the particles show very small magnetization per volume, for example the particle characteristics designed to have average diameter of equal to or larger than 30 nm. The magnetization decrease of ferrite particles due to their miniaturization involves nonmagnetic surface layer. The fraction of the nonmagnetic surface layer increases when the diameter becomes small and result in rapid decrease of magnetization. As the particle size become small, the particles show single magnetic domain structure in the first place, and then fluctuation of the magnetization direction due to thermal fluctuation effect occurs.

In each application of the polymer coated ferromagnetic particles, the surfaces of the ferromagnetic particles are desired to be covered sufficiently with polymer. In addition, the polymer coating is desired to form bonding between polymer and ferromagnetic particles such as ferrite particles tight enough to avoid dissociation during use of the particles.

Core ferromagnetic particles are desired to have large magnetization to obtain polymer coated ferromagnetic particles with good response to magnetic field for handling. However, the particles tend to flocculate strongly as a result of strong magnetic interaction between particles when the ferromagnetic particles have large magnetization. Therefore, it was difficult to obtain sufficiently dispersed fine polymer coated ferromagnetic particles in which each particle is coated with polymer.

When the ferromagnetic particles are ferrite particles, the particle size is desirable to be 20 nm or more, and is more desirable to be 30 nm or more for obtaining sufficient magnetization. However, it is very difficult to disperse ferrite particles having particle diameter of 30 nm or more, because the attractive force between the particles is strong enough. Then it has been difficult to obtain sufficiently dispersed fine ferrite particles in which each particle was coated with polymer. For this reason, known polymer coated ferrite particles having an extent of diameters of about 10 nm or less are limited to particles the type in which each aggregate of particles is coated as a whole, and the type in which coated particles of very small diameter ferrite particles with very weak magnetic aggregating force having very weak response to magnetic field because the particles have very small magnetization.
[Patent reference 1] JP-A 2002-090366 (KOKAI)
[Patent reference 2] WO 03/066644 A1
[Patent reference 3] JP-A 2003-513093 (KOKAI)
[Non-patent reference 1] Scientific and Clinical Applications of Magnetic Carriers, Plenum Press 1997

### DISCLOSURE OF THE INVENTION

This invention presents manufacturing method for obtaining polymer coated ferromagnetic particles having small particle size and showing good response to magnetic field, solving the existing technical problem of dispersing and polymer coating ferromagnetic particles having strong attractive force, and presents highly dispersed polymer coated ferromagnetic particles showing good response to magnetic field.

The method of manufacturing polymer coated ferromagnetic particles of the present invention comprises an emulsifying process of obtaining emulsified liquid by mixing hydrophobic ferromagnetic particles having average diameter of 20 to 300 nm and monomer liquid containing monomer and surface activating agent carrying nonionic hydrophilic group with water and emulsifying, and emulsion polymerization process of conducting emulsion polymerization adding initiator causing radical addition polymerization to the emulsified liquid. To obtain the hydrophobic ferromagnetic particles in the present invention of manufacturing polymer coated ferromagnetic particles, an hydrophobizing process of adsorbing hydrophilic group of hydrophobizing agent having hydrophilic group and hydrophobic group to the particles having hydrophobic character can further be comprised.

As a result of concentrated research works of the present invention, they attained at the present invention finding that sufficiently dispersed polymer coated ferromagnetic particles could be obtained by providing hydrophobic character to ferromagnetic particles and the ferromagnetic particles having hydrophobic character were mixed in water and emulsifying to obtain emulsion liquid with surface activating agent having nonionic hydrophilic group and by emulsion polymerizing the emulsion liquid, even if the ferromagnetic particles had average diameter of 20 nm or more and the particles have magnetic adhesive force showing ferromagnetic characteristics. When the hydrophilic ferromagnetic particles were ferrite particles, it was found that sufficiently dispersed polymer coated ferrite particles can be obtained using this method, even if the ferromagnetic particles have average particle diameter of 30 nm or more. When average diameter of the ferromagnetic particles is larger than 300 nm, the character of the ferromagnetic particles at the emulsifying process changes significantly. Therefore, it is found that the method of this invention is suited to coating ferromagnetic particles having average particle diameter of 300 nm or less. The monomer described above can be monomer in liquid state in itself or monomer liquid as a solution of organic solvent. The monomer can also be a monomer mix containing initiator and so on.

Nonionic surface activating agent can be used as the surface-activating agent in the present invention. It is found that especially good result is obtained by using nonionic surface activating agent and ionic activating agent in combination.

The polymer coated ferromagnetic particles of the present invention can be particles having average diameter of from 25 to 400 nm, comprising hydrophilic ferrite particles having average particle diameter of 20 to 300 nm, hydrophobizing agent having hydrophilic group and hydrophobic group to provide hydrophobic characteristics to the ferrite particles adsorbing to the ferrite particles through the hydrophilic group of the agent, and polymer coat to adsorb to the hydrophobizing agent and coat the hydrophobized ferrite particles.

Polymer coated ferromagnetic particles that are used in organic solvent according to the present invention are polymer coated ferromagnetic particles durable to organic solvent as explained above. The magnetic solid phase carrier particles for combinatorial chemistry, the magnetic solid carrier particles for affinity chromatography of protein, peptide, or nucleic acid, and magnetic solid phase carrier particles for chemical synthesis of peptide or nucleic acid of the present invention are characterized by using these polymer coated ferromagnetic particles.

The magnetic solid carrier particles for combinatorial chemistry, the magnetic carrier particles for affinity chromatography, and the magnetic carrier particles for chemical synthesis having such construction can have significant solvent durability resistive to the dissolution to the solvents and resistive to the polymer desorption from the particles keeping the polymer coating stable even if the particles are immersed into organic solvents including methanol, ethanol, isopropanol, tetrahydrofuran, acetonitrile, ethyl acetate, dioxane, N,N-dimethylformamide, dimethylsulfoxide, acetone, diethylether, toluene, dichloromethane, chloroform and hexane. The particles can have an advantage of using in organic solvent for magnetic separation since the particles having magnetization are solvent resistive. The particles, therefore, can be used as carrier that can work as stable and quick separation process of combinatorial chemistry, affinity chromatography and chemical synthesis.

As a result of the present invention, a problem of difficulty for dispersing relatively large size ferromagnetic particles having strong cohesive force is solved, and homogeneous and stable polymer coating of ferromagnetic particles such as ferrite particles sufficiently dispersed to monodispersed state or almost monodispersed state can be realized. The particles can be used for various applications including bio-separation in fields of bio-science and clinical medicine, since the polymer coated ferromagnetic particles obtained applying the present invention can have very small particle diameter, can have stable polymer coating and can show good magnetic field response. In each industrial field, for example, significant characteristics increase in magnetic colloid, magnetic ink and magnetic paint can be obtained due to the distinctive character showing good response to magnetic field and stability.

### BRIEF DESCRIPTION OF DRAWINGS

FIG. 1 shows a process flow of an embodiment for manufacturing polymer coated ferromagnetic particles according to the present invention.

FIG. 2 shows an embodiment of the emulsifying process shown in FIG. 1 as a flow.

FIG. 3 shows an embodiment of the process for providing hydrophilic character shown in FIG. 2 as a flow.

FIG. 4A is another embodiment of the emulsifying process shown in FIG. 1 as a flow.

FIG. 4B is a yet other embodiment of the emulsifying process shown in FIG. 1.

FIG. 5 schematically shows an embodiment of the process for coating polymer to ferromagnetic particles partially.

FIG. 6 schematically shows an embodiment of the process for coating polymer to ferromagnetic particles partially.

FIG. 7 schematically shows an embodiment of polymer coated ferromagnetic particles.

FIG. 8 shows an electron microscope photographs for an embodiment of polymer coated ferromagnetic particles.

### DESCRIPTION OF SYMBOLS

100 . . . hydrophobizing process, 110 . . . ferromagnetic particles, 120 . . . hydrophobizing material, 130 . . . hydrophobic ferromagnetic particles, 200 . . . emulsifying process, 202 . . . first hydrophilic ferromagnetic particles, 203 . . . first process for providing hydrophilic characteristics, 204 . . . second hydrophilic ferromagnetic particles, 205 . . . the second process for providing hydrophilic characteristic, 210 . . . process for providing hydrophilic characteristics, 211 . . . nonionic and ionic surface activating agent, 212 . . . hydrophilic ferromagnetic particles, 213 . . . nonionic surface activating agent, 214 . . . ionic surface activating agent, 220 . . . monomer adding and mixing process, 221 . . . monomer liquid, 222 . . . monomer emulsifying process, 223 . . . monomer emulsion liquid, 224 . . . mixing process, 230 . . . hydrophobic initiator, 240 . . . sonication, 250 . . . water, 260 . . . emulsion liquid, 300 . . . emulsion polymerization process, 310 . . . water, 320 . . . sonication, 330 . . . heating, 340 . . . hydrophilic initiator, 350 . . . rinsing process, 360 . . . polymer coated ferrite particles, 360a . . . polymer coat, 710 . . . epoxy group, 720 . . . amino group, 730 . . . ethylene glycol glycidylether (EGDE).

### BEST MODE FOR CARRYING OUT THE INVENTION

Embodiments of the present invention will be explained specifically below with reference to figures. The description given below shows as examples specific embodiments of the present invention and the description does not limit the scope of the present invention.

### (1) Polymer Coating Process for Ferromagnetic Particles

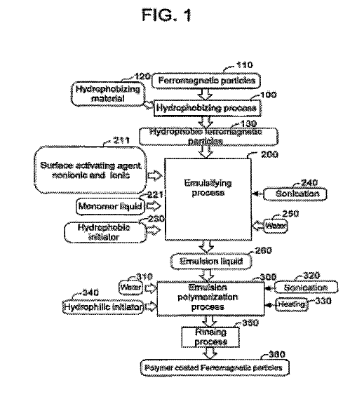

FIG. 1 shows a process flow of an embodiment for manufacturing polymer coated ferromagnetic particles. To begin with the hydrophobizing process 100 of FIG. 1, the hydrophobic ferromagnetic particles 130 are obtained adding hydrophobizing material 120 such as aliphatic acid to adsorb to the ferromagnetic particles 110. At the emulsifying process 200, nonionic surface activating agent having nonionic hydrophilic group such as polyethylene oxide (PEO) chain and ionic surface activating agent having cationic hydrophilic ion group 211 such as primary amine or anionic group such as carboxyl group are mixed with the hydrophobic ferromagnetic particles 130, and monomer liquid 221 containing monomer carrying functional group having potential of radical addition polymerization such as styrene and glycidyl metacrylate (GMA), for example, with organic solvent as needed, is added with water 250 to the hydrophobic ferromagnetic particles. The mixture is then emulsified and emulsion-polymerized. Especially good result is obtained by controlling polymer-coating formation to the ferromagnetic particles of the emulsion polymerization process. In the monomer liquid, hydrophobic initiator 230 such as azobisisobutylonitrile (AIBN) is added as needed. To the monomer liquid 221, cross-linking agent such as divinylbenzene for example is added as needed. Adequate quantity of water 250 is added as needed. Emulsion liquid 260 is obtained mixing and emulsifying the mixture. For the emulsification at the emulsification process 200, sonication 240 of giving ultra sound vibration is proved useful.

At the next process of emulsion polymerization process 300, the emulsion liquid 260 after sonification is heated at temperature equal to or lower than 100° C., desirably at 50 to 90° C., and more desirably at 6 to 80° C., and then hydrophilic initiator is added to polymerize the monomer in the emulsion. In the emulsion polymerization process, it is inferable that the polymerization of monomers proceeds various interactions between monomers inside an emulsion particle, and outside the emulsion particle and monomers between another emulsion particle. The length of the polymerization time period can be set arbitrarily and it may proceed polymerization for about 24 hours.

After removing the surface activating agent by rinsing the polymerized emulsion particles, polymer coated ferromagnetic particles can be obtained

The emulsifying process 200 explained above and shown in FIG. 1 can be proceeded dividing so as to divide in two processes of process for providing hydrophilic character 210 and monomer adding and mixing process 220 to be processed sequentially.

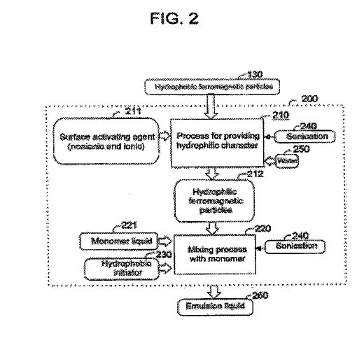

At the process for providing hydrophilic character 210 shown in FIG. 2, hydrophilic ferromagnetic particles 212 dispersion liquid is obtained by adding and adsorbing surface activating agent 211 containing nonionic surface activating agent and ionic surface activating agent to hydrophobic ferromagnetic particles 130 and further by dispersing, supplying adequate quantity of water 250 as needed. The monomer adding and mixing process 220 can be constructed such that the emulsion liquid 260 is obtained by adding, mixing and emulsifying the monomer liquid 221 explained above to the dispersion liquid of hydrophilic ferromagnetic particles 212. In these processes, sonication 240 providing ultrasonic vibration can be used.

In these way, polymer coated ferromagnetic particles with desirable polymer coating formed at each particle of the hydrophobic ferromagnetic particles 130 can be obtained as a result of polymerizing at the emulsion polymerization process the emulsified emulsion liquid 260.

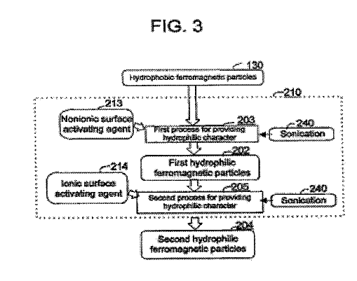

The process for providing hydrophilic character 210 shown in FIG. 2 is desirable to be divided to the two processes. The first process for providing hydrophilic character 203 is a process for obtaining the first hydrophilic ferromagnetic particles 202 adsorbing nonionic surface activating agent 213 to the hydrophobic ferromagnetic particles 130. The second process for providing hydrophilic character 205 is a process for obtaining the second hydrophilic ferromagnetic particles 204 adsorbing ionic surface activating agent 214 to the first hydrophilic ferromagnetic particles as shown in FIG. 3.

In this way, dispersion degree of the ferromagnetic particle having hydrophilic character can be increased and the highly dispersed state can be stabilized. Polymer coated ferromagnetic particles having further desirable dispersion degree, and further good polymer coating with sharp particle size distribution can be obtained by proceeding the emulsion polymerization of the emulsion liquid 260 prepared using the ferromagnetic particles having hydrophilic character.

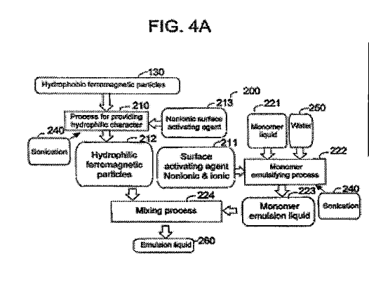

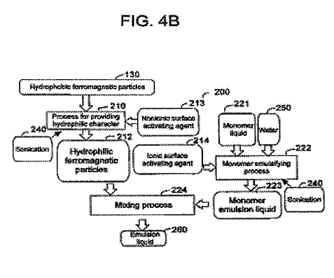

The emulsifying process 200 shown in FIG. 1 can have a constitution as shown in FIG. 4A. At the emulsifying process 200 shown in FIG. 4A, the dispersion liquid of the ferromagnetic particles having hydrophilic character 212 is obtained by providing hydrophilic character to the hydrophobic ferromagnetic particles adding water solution of surface activating agent 213 having nonionic hydrophilic group to hydrophobic ferromagnetic particles 130. At the monomer emulsifying process 222, on the other hand, monomer emulsion liquid 223 is obtained adding water solution of nonionic surface activating agent and ionic surface activating agent 211 to the monomer liquid 221 and mixing and emulsifying adding water 250 as needed. The process can be constructed such that the emulsion liquid 260 is obtained by mixing the dispersion liquid of the ferromagnetic particles having hydrophilic character 212 and the monomer emulsion liquid 223 at the mixing process 224. In each of these emulsifying process, sonication 240 giving ultrasonic vibration to liquid can be applied.

As a result of emulsion polymerizing the polymerized liquid 260 in this way, polymer coated ferrite particles having desirable polymer coating to each of the hydrophobic ferromagnetic particles 130 and having desirable dispersion characteristics can be obtained.

As shown in FIG. 4B, nonionic surface activating agent 213 is used similar to the case for FIG. 4A at the process for providing hydrophilic character 210 in the emulsifying process 200 of providing hydrophilic character to the hydrophobic ferromagnetic particles 130. For emulsifying monomer liquid 221, on the other hand, the ionic surface activating agent 214 can be used. In this way, as a result of emulsion polymerizing the polymerized liquid 260, polymer coated ferrite particles having desirable polymer coating to each of the hydrophobic ferromagnetic particles 130 and having desirable dispersion characteristics can be obtained.

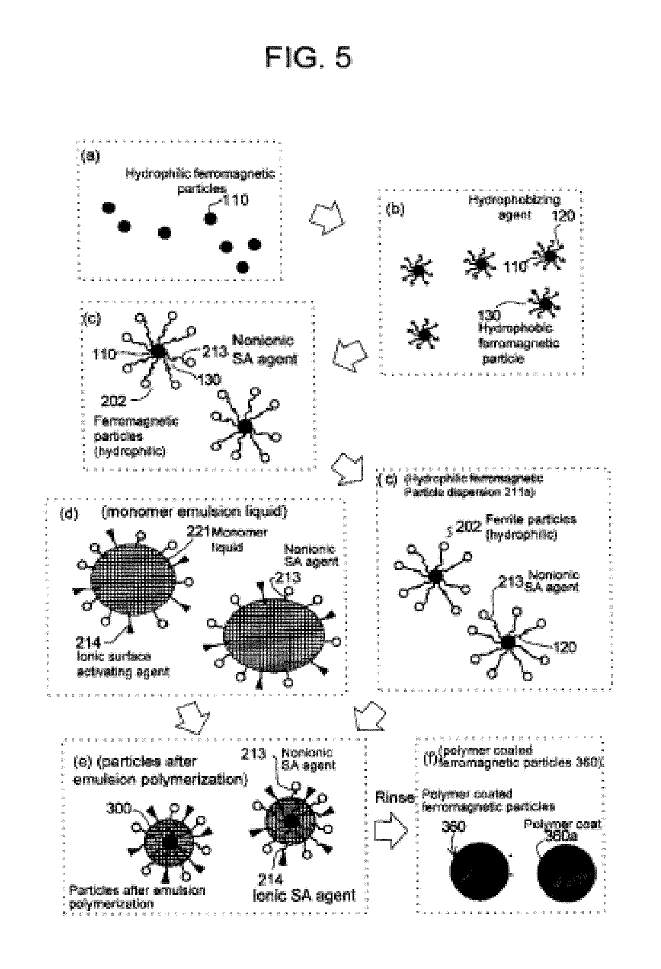

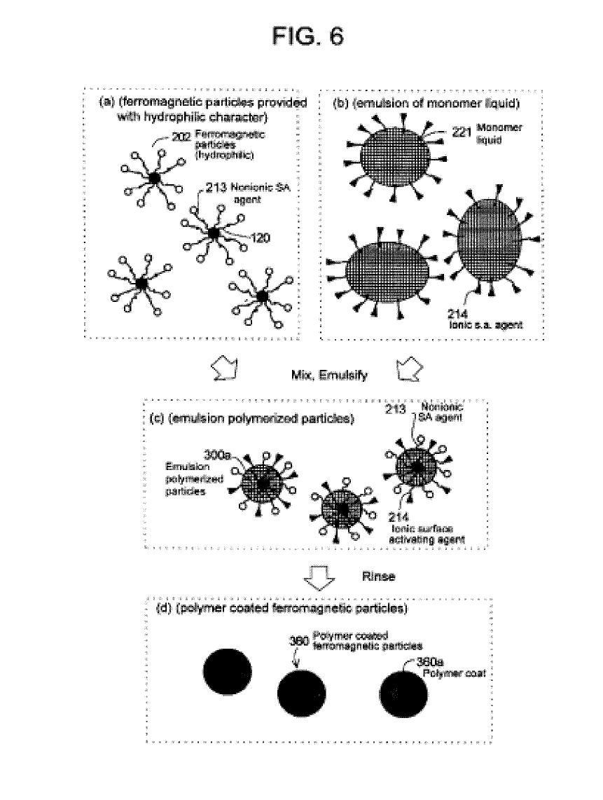

FIG. 5 and FIG. 6 are figures schematically showing an embodiment for polymer coating to ferromagnetic particles.

Processing the hydrophilic ferromagnetic particles 110 shown (a) in FIG. 5 with hydrophobizing material 120 such as aliphatic acid as shown (b) in FIG. 5 to hydrophilic ferromagnetic particles 110 shown (a) in FIG. 5, hydrophobic ferromagnetic particles 130 hydrophobized by using hydrophobizing material 120 are obtained.

Dispersion liquid in which hydrophilized ferromagnetic particles 202 are dispersed in water as shown schematically (a) in FIG. 5 is obtained by immersing and dispersing the hydrophobic ferromagnetic particles 130 in water solution of nonionic surface activating agent 213.

As shown (c) in FIG. 5, dispersion liquid of hydrophilic ferromagnetic particles dispersing ferromagnetic particles having hydrophilic ferromagnetic character 202 and emulsion liquid 221 shown (d) in FIG. 5 are mixed, i.e., the emulsion liquid 221 prepared by mixing monomer liquid having water as continuous phase using nonionic surface activating agent 213 and ionic surface activating agent 214. The mixture is emulsified giving ultrasonic vibration as sonication. The obtained emulsion is then heated up to 50 to 90° C., desirably to 60 to 80° C., and emulsion was polymerized adding and mixing initiator. Then emulsion polymerized particles 300a shown schematically (e) in FIG. 5 are obtained. After removing nonionic surface activating agent 213 and ionic surface activating agent 214 from the emulsion polymerized particles 300a by rinsing, polymer coated ferromagnetic particles 360 coated with polymer coat 360a as shown schematically (f) in FIG. 5 are obtained. According to this procedure, polymer coated ferromagnetic particles having desirable dispersibility, coating quality and sharp particle diameter distribution can be obtained.

The emulsion polymerization can be performed using nonionic surface activating agent solution 213 to obtain hydrophilic property of the hydrophobic ferromagnetic particles 130 at one hand as shown (a) in FIG. 6, and using only surface activating agent 214 having hydrophilic group do obtain monomer emulsion liquid 221, at the other hand, as shown (b) in FIG. 6. According to this procedure, polymer coated ferromagnetic particles 360 having desirable dispersibility, polymer coat 360a with desirable coating quality and sharp particle diameter distribution can be obtained.

### (2) Ferromagnetic Particles

As the ferromagnetic particles applied to these ferromagnetic particles, magnetite (Fe₃O₄), maghemite (γ-Fe₂O₃), and particles having intermediate composition of these particles can be used. In addition, ferrite particles in which the characteristics of the particle are controlled adequately for each purpose replacing a part of Fe elements in magnetite particles by other element such as Li, Mg, Mn, Co, Ni, Cu and Zn, can be used. For example, larger magnetization can be obtained by replacing part of Fe in ferrite such as magnetite by Mn or Zn. Further, metal particles having ferromagnetic characteristics such as Fe, Co, and Ni can be applied instead of ferrite particles. Particles having larger saturation magnetization per volume can be obtained by using metal alloys or inter-metallic compounds. Inorganic ferromagnetic particles can be applied utilizing their advantageous characteristics. It is essentially important to achieve resistance to corrosion for applying these particles. When Fe particles are used, for example, the surfaces are desirable to be covered with stable oxide such as magnetite. Polymer coating of these Fe particles can be obtained using the method similar to the case for polymer coating to magnetite particles.

For the purpose of handling polymer coated ferromagnetic particles easily using magnetic field, the particles are desirable to show ferromagnetic character at temperature at which the coated particles are used. When the ferromagnetic particles are ferrite particles, for example, the particle average diameter of the ferrite particles is desirable to be 30 nm or more. From the point of ensuring sufficient dispersion stability of the ferrite particle dispersion liquid, however, the particle average diameter of the ferrite particles is desirable to be 300 nm or less and more desirable to be 100 nm or less. The average diameter of ferrite particles in the present invention described above is obtained calculating arithmetic average of the measured diameter values of 500 particles obtained by use of transmission electron microscope photographs.

### (3) Material for Providing Hydrophobic Character

As the agent material for providing hydrophobic character to ferromagnetic particles having hydrophilic character, materials adsorbing tightly at the surface of the ferromagnetic particles and providing hydrophobic character to ferromagnetic particles with abrasion resistive at treatments such as water treatment are desirable. Materials having hydrophilic group such as carboxyl group and phosphoric group, such as aliphatic acid and phosphatide showing tight adsorption character, for example, are desirable for this purpose. The aliphatic acid can be adsorbed to ferromagnetic particles by contacting the aliphatic acid water solution to the ferromagnetic particles and adjusting pH value of the solution.

The material for providing hydrophobic character is desirable to carry functional group having capability of radical polymerization reaction. When the material for providing hydrophobic character carries functional group having capability of radical polymerization, co-polymerization between the material for providing hydrophobic character and the monomer. Then sufficient and stable polymer coats can obtained that coat the ferromagnetic particles. As the atomic groups having capability of radical polymerization, atomic groups having double bond between carbon atoms at the terminal such as vinyl group (CH₂═CH—), methacryl group (CH₂═C(CH₃)—CO—) can be cited.

When aliphatic acid carrying double bond between carbon atoms at the terminal of hydrophobic group and carrying no other double bond between other carbon atoms is applied as the material for providing hydrophobic character, the number of carbons is desirable to be from 6 to 18. The number of carbons more desirable to be from 8 to 15, and further more desirable to be from 10 to 15. Of these the aliphatic acids, 10-undecenoic acid is one of the materials for providing hydrophobic character applied in the present invention. When aliphatic acid carrying double bond between carbon atoms at the terminal of hydrophobic group side and further carrying other double bond between carbon atoms as the material for providing hydrophobic character to obtain hydrophobic ferromagnetic particles, the number of carbons is desirable to be from 6 to 20, more desirable to be from 10 to 20, further desirable to be from 12 to 18.

The material for providing hydrophobic character applied in the present invention is desirable to be material having atomic groups that form bond with hydroxyl group at the surface of ferromagnetic particles. As such material, metal alkoxides such as aluminum coupling agent, silane coupling agent and titanium coupling agent, acid chloride, carboxylic acid anhydride, and organic compounds carrying isocyanate groups can be cited.

These materials for providing hydrophobic character are also desirable to carry atomic groups having capability of radical polymerization reaction with the radical polymerizing monomers. As explained already, since the materials providing hydrophobic character having functional group capable of radical polymerization reaction of the materials for providing hydrophobic character provides desirable and stable polymer coating to the ferromagnetic particles.

In recent years, methods of synthesizing ferrite particles in organic solvents, represented by polyol reduction method and inverse micelle method, have been generally used as described in Sun S. et al. J. Am. Chem. Soc. (2004), 126(1); 273-279, and Jongnam P et al. Nature Mater. (2004) 3 891-895. Ferrite particles using these methods show hydrophobic character having reactant material adsorbed already at the surface of the ferrite particles after the synthesis reaction. Of course the ferrite particles synthesized using these methods can be available as the hydrophobic ferrite particles of the present invention.

### (4) Surface Activating Agent

In the present invention, nonionic surface activating agent and ionic surface activating agent can be used as the surface activating agent for providing hydrophilic character by adsorbing the agent to hydrophobic ferromagnetic particles that are provided with hydrophobic character for providing the agent, for emulsifying adding emulsifying monomer liquid, and for polymerizing the emulsion. As the surface activating agent for the purpose, adequate combination of nonionic surf ace activating agent and ionic surface activating agent can be used. Instead of using the adequate combination of nonionic surface activating agent and ionic surface activating agent, surface activating agent having both nonionic functional group and ionic functional group in one molecule of surface activating agent can be used.

As the nonionic surface activating agent having nonionic hydrophilic group used in the present invention, various surface activating agent having polyethylene oxide (PEO) chains, or the one having PEO chains with partially replaced by oxypropylene or oxybutylene chains can be desirably applied. As the surface activating agents having nonionic hydrophilic group, polyoxyethylene alkylether (product name: Emulgen (supplied by Kao Co.)) and nonylphenol polyethoxylate Trinton X-405 can be used.

These nonionic surface-activating agents can be used as water solution at a concentration of 70%, for example. The quantity of addition is therefore desirable to be at the rate of 0.012 g or more per 1 g of monomer. The quantity is more desirable to be at the rate of 0.047 g or more per 1 g of monomer. Too much addition of surface activating agent, however, is not desirable because polymer-coating formation becomes difficult. Therefore, the quantity of addition thereof is desirable to be 0.23 g or less and is more desirable to be 0.1 g or less per 1 g of monomer.

Of ionic surface activating agents, various surface activating agents having anionic hydrophilic group such as sulfonic acid group, sulfuric acid group, phosphoric acid group, or polyphosphoric acid group can be used in addition to the surface activating agent having carboxyl group such as aliphatic acid. As surface activating agent having carboxyl group, aliphatic acids carrying 11-15 carbons with straight chain alkyl group can be desirably used. As surface activating agents having cationic hydrophilic atomic group, various surface activating agents having amino group such as long chain primary amine, secondary amine, various tertiary amine, and quaternary ammonium ion. Similarly, surface activating agents having unsaturated bond in the alkyl chain can also be used desirably. The hydrophobic group of the ionic surface activating agent is desirable to be a long chain having prescribed length or more to maintain micelles of the emulsion stable in the process of emulsion polymerization process. For example, the molecule of linear chain type primary amine CₙH₂ₙ₊₁NH₂ is desirable to have long chain of n being 10 or more, and the molecule is further desirable to have chain of n being 11 or more. On the other hand, when n is too large, the function as the agent for forming emulsion is not sufficient. Therefore, the n value is desirable to be 17 or less, and the n value is more desirable to be 15 or less, such as the molecule, for example, aminoundecane.

It is difficult to determine the quantity of adding the ionic surface-activating agent, because the quantity depends on the purpose and the size of the polymer coated ferromagnetic particles. Here, only outline of the desirable quantity is described. An example of the desirable quantity is 1.5 μmol or more, and the more desirable quantity is 6 μmol or more. On the other hand, the quantity of the ionic surface-activating agent is desirable to be less than 50 μmol or equal to 50 μmol, and is more desirable to be less than 16.5 μmol or equal to 16.5 μmol.

### (5) Monomer and Organic Solvent for Forming Monomer Solution

The most suitable monomer used for the present invention can be chosen from various monomers carrying functional group having capability of radical polymerization reaction on its purpose. As these monomers carrying functional group having capability of radical polymerization reaction: aromatic vinyl compounds including styrene, .alpha.-methyl styrene, o-vinyl toluene, m-vinyl toluene, p-vinyl toluene and divinyl benzene; unsaturated carboxylic acids including (meta) acrylic acid and crotonic acid; (meta) acrylates including methyl(meta) acrylate, ethyl(meta) acrylate, n-propyl(meta) acrylate, i-propyl(meta) acrylate, n-butyl(meta) acrylate, t-butyl(meta) acrylate, n-hexyl(meta) acrylate, 2-ethylhexyl(meta) acrylate, (poly)ethylene glycol di(meta) acrylate, (poly) polypropyleneglycol di(meta) acrylate, trimethylol propane tri(meta) acrylate, glycidyl(meta)acrylate; vinyl cyanide compounds including (meta) acrylonitrile, vinylidene cyanide; Vinyl halide compounds including vinyl chloride, vinylidene chloride, vinyl floride, vinylidene floride, tetra fluoroethylene can be cited. Of these monomers, aromatic vinyl compounds and (meta) acrylates of these monomers can especially be used desirably. These monomers can be used as one type or as mixture of plural types. At least one type of these monomers used is desirable to have functional group that can bond to other material placed at the polymer coated surf ace after polymer coating. When a combination of styrene and glycidylmethacrylate (GMD) is used at the surface of the formed coating, glycidyl group (epoxy group) can be placed at the surface of the coating, for example and various compounds including biologically active substance through the functional group.

Various organic solvents can be used as organic solvents for solving these monomers to obtain monomer solution. For example, alkanes having 10-20 carbon atoms per molecule, diethyl ether and so on can be used. Diethyl ether shows hydrophobic character due to the two ethyl groups of the molecule, although the ether has hydrophilic ether bond. Adding surface activating agent in water, emulsion particles of diethyl ether can be obtained. So, the diethyl ether can desirably be used as organic solvent for monomer solution liquid. The monomer for use can be solved into organic solvent in a predetermined composition ratio and can be used as monomer mix adding hydrophobic initiator to the solution as needed. Composition of the monomer mix can be controlled such that desirable polymer coated ferromagnetic particles are formed by emulsion polymerization.

### (6) Initiator and Cross Linking Agent

In order to carry out radical polymerization of the monomer, the emulsion of the monomer mix is heated and hydrophilic initiator is added and mixed to the continuous water phase of the emulsion. Hydrophobic initiator also can be used by adding and mixing to the monomer.

As water solve hydrophilic initiators, peroxides including benzoyl peroxide, potassium peroxodisulfate (KPS), and ammonium peroxodisulfate (APS); and water soluble azo compounds including V-50 (product name, supplied by WAKO Co., (2,2′-Azobis(2-methylpropionamidine) dihydrochloride, (2,2′-Azobis(2-amidinopropane) dihydrochloride)) can be used. As hydrophobic initiators, various azo-compounds such as azobisisobutyronitrile (AIBN) can be used.

As other initiators, azo-compounds including VA-080 (product name, by WAKO Co., (2,2′-Azobis(2-methyl-N-[1,1-bis(hydroxymethyl)-2-hydroxyeth yl]propionamide), VA-085 (product name, by WAKO Co., (2,21-Azobis{2-methyl-N-[2-(1-hydroxybutyl)]-propionamide}), VA-086 (product name, by WAKO Co., (2,2′-Azobis[2-methyl-N-(2-hydroxyethyl)-propionamide]), VA-057 (product name, by WAKO Co., (2,2′-Azobis (N-(2-carboxyethyl)-2-methyl-propionamidine, (2,2′-Azobis{2-[N-(2-carboxyethyl)amidino]propane}), V-501 (product name, by WAKO Co., (4,4′-Azobis(4-cyanovaleric acid), (4,4′-Azobis(4-cyanopentanoic acid)), VPE-0201 (product name, by WAKO Co.), and VPE-0601 (product name, by WAKO Co.) can be used.

As cross-linking agent, at least either one of cross-linking agents including divinyl benzene, triarylamine, 1,3,5-triacryloil-hexahydro-s-triazine, trimecin acid triaryl, and ethyleneglycol dimethaclilate can be used.

### (7) Polymer Coated Ferromagnetic Particles

Several examples of the polymer coated ferromagnetic particles obtained in these procedures are explained.

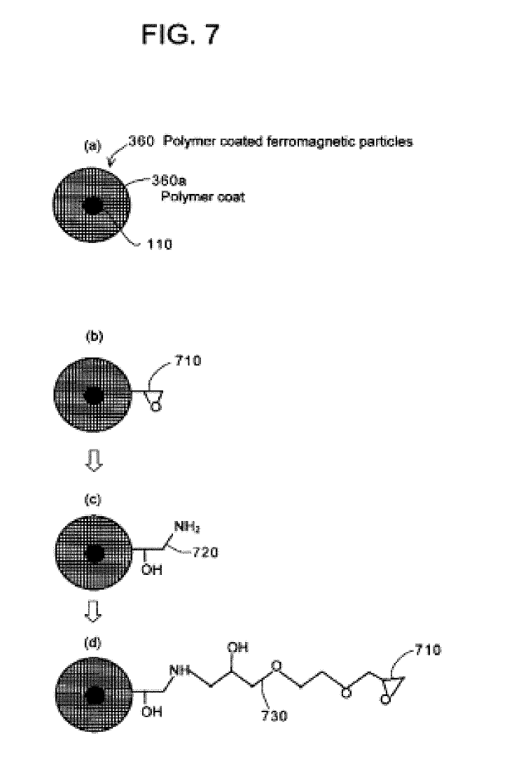

FIG. 7 illustrates schematically an embodiment of the polymer coated ferromagnetic particles according to the present invention. The (a) of FIG. 7 is a figure showing the polymer coated ferromagnetic particles 360a, monomer forming coating and polymerizing ferromagnetic particles 110 through material providing hydrophobic character adsorbed to the hydrophilic ferromagnetic particles.

The (b) of FIG. 7 shows 10-undecenoic acid adsorbed to the hydrophilic ferromagnetic particles 110 and co-polymerized with styrene and glycidyl metacrylate coating the hydrophilic ferromagnetic particles 110. At the surface of the coated particles, many terminals of epoxy group 710 are placed. Actually, many terminals of epoxy group 710 are existed on the surface of each particle, schematically only one terminal is shown for simplicity. The (c) of FIG. 7 shows one of the particles opening the glycidyl group (epoxy group) and providing hydroxy group terminals and amino group 720 terminals by treating the particles shown (b) in FIG. 7 with ammonium. The (d) of FIG. 7 shows the particles providing spacer 730 by bonding monoethylene glycol glycidylether or polyethylene glycol glycidylether (EGDE) molecule to the amino group 720 shown (c) in FIG. 7. The particles can be applied as carrier of affinity chromatography. Providing epoxy group 710 at the top end of the spacers 730 that bond biological molecules, The particles can easily bond various molecules avoiding steric hinderance.

When the particles of the polymer coated ferromagnetic particles are ferrite particles, the particle can be used for various applications by choosing the diameter size of within 25-400 nm because the magnetization can be obtained at suitable values for handling using magnetic field. For use as media for performing magnetic separation of biological molecules, for example, it is desirable that the average ferrite particle diameter of 30-100 nm and the average polymer coated particle diameter of 35-150 nm with weight ratio of the magnetic particles to 1/10 or more, and more desirable to be ¼ or more to ensure magnetic attractive force of the particles. These polymer coated ferromagnetic particles are desirable to have uniformity in their particle sizes. When the particle size uniformity are lost, measurement results using the particles loose their reproducibility. For this reason, standard deviation of the particle distribution is desirable to be no more than 50 percent of the average particle diameter and more desirable to be no more than 10 percent. The average diameter is arithmetic average diameter averaged to number of the particles.

The organic solvent durability of the polymer coating can be increased by cross-linking the polymer using cross-linking agent. The organic solvent durability can be increased further by applying the EGDE modification to the polymer coating.

These particles according to the present invention can be used as media for magnetic handling in biological science application, medical applications and various industrial application, because each particle of these polymer coated ferromagnetic particles can be separated each other having uniform particle size and shows desirable response to magnetic field. Many applications of the particles making best use of the particle characteristics of the particles are expected such as magnetic colloid application showing excellent magnetic field response and stability compared with magnetic colloids using known polymer coated ferromagnetic particles.

It is found that the polymer coated ferromagnetic particles according to the present invention can be fine particles with excellent organic solvent durability. The polymer coating can be not solved by organic solvents and can keep their shapes even if the particles are immersed in organic solvents. The solvent durability can increase by cross-linking the polymer. Increase of the organic solvent durability can also be obtained by providing spacer such as EGDE.

The polymer coated ferromagnetic particles having such constitution can be used for various applications such as carrier particles of affinity chromatography for chemical materials including proteins, peptides, nucleic acid and drugs, magnetic solid phase magnetic carrier particles for combinatorial chemistry, as the magnetic solid carrier particles for of chromatography, and as magnetic solid phase carrier particles for chemical synthesis of peptide or nucleic acid. For example when the particles are used as magnetic carrier of affinity chromatography, the availability of the particles in organic solvent allows bonding water insoluble compounds to the coating of the ferromagnetic particles quantitatively as ligands in organic solvent.

As a method for detecting biological molecules including nucleic acids, methods using a magnetic sensor are known. As the magnetic sensors, giant magnetoresistance effect device (GMR), tunnel magnetoresistance effect device (TMR), hall sensor, and SQUID type flux meter are cited. Bio-molecules are fixed on the device and are also fixed on the magnetic particles. A magnetic signal can be obtained only when there are interactions between these bio-molecules. The polymer coated ferromagnetic particles according to the present invention can be spherical particles with narrow particle size distribution. Since the particles can be homogeneous and each particle can carry similar number of ferromagnetic particles, the particles are useful as an excellent magnetic marker to bio-molecules of a magnetic sensor system.

### EXAMPLE 1

100 μl of 10-undeconoic acid is added to 150 mg of ferromagnetic ferrite particles having average particle diameter of 40 nm obtained by precipitating from water solution and the 10-undecenoic acid was adsorbed in saturation to the ferromagnetic particles. Remained 10-undecenoic acid that did not adsorbed to the particles was eliminated by rinsing. Then hydrophobic ferromagnetic particles were obtained.

Nonionic surface activating agent water solution containing 0.3 g of Emulgen 1150S-70 (produced by KAO Co.) having chemical formula I and carrying PEO chain was added to the hydrophobic ferromagnetic particles and adsorbed the nonionic surface activating agent to the ferromagnetic particles having hydrophilic character, and ultrasonic vibration was applied as sonication. Then colloidal water liquid of ferrite particles having hydrophilic character was obtained.

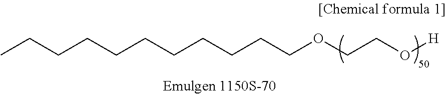

The water solution dissolving 10 μl of ionic surface activating agent aminoundecane solved in 56 μl of 1 M HCl was added to the colloidal water liquid of ferrite particles having hydrophilic character. Then a state of coexisting nonionic surface activating agent and ionic surface activating agent at the surface of ferrite particle colloid having hydrophilic character was established.

Then emulsion liquid was obtained by adding monomer mix containing 2.7 g of styrene (monomer), 0.3 g of GMA (glycidyl metacrylate, monomer), 0.025 g of AIBN (azobisisobutylonitrile, initiator), 0.08 g of DVB (divinylbenzene, cross linking agent) and 2.5 g of diethylether to the colloidal liquid and applying ultrasonic vibration as sonication.

Then water was added to the emulsion liquid such that the total liquid quantity of 125 g and applied ultrasonic vibration of sonication. The liquid was then heated under mixing at rotation speed of 350 rpm. When the temperature of the liquid attained at 70° C. about 20-30 minutes after beginning of the heating, water soluble initiator V-50 (product of WAKO PURE CHEMICAL Co.) was added and polymerization reaction was proceeded for 12 hours.

Polymer coated ferrite particles were obtained after rinsing the emulsion polymerized particles. As a result of electron microscope observation using a transmission electron microscope, the obtained polymer coated ferrite particles were monodispersed particles carrying 1-3 ferrite particles inside each particle having average particle diameter of 163 nm with the diameter standard deviation of 20 nm.

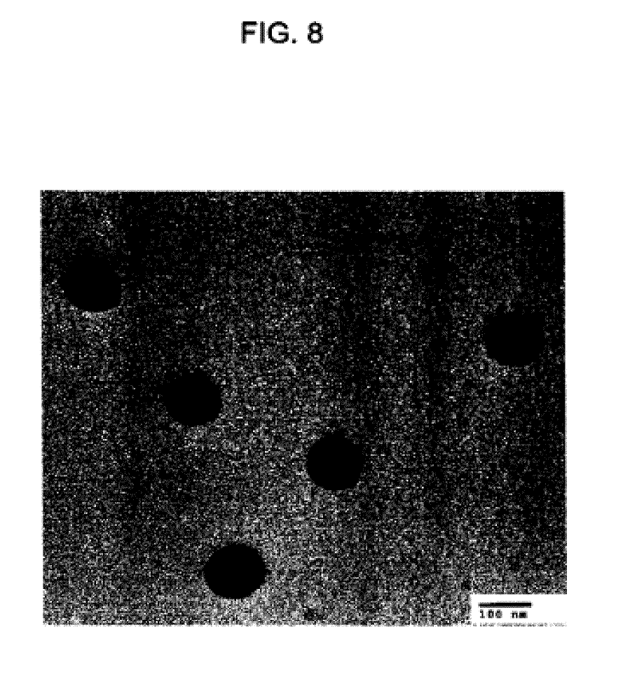

FIG. 8 shows an example of electron microscope photographs for the prepared polymer coated ferrite particles using this preparation method.

The polymer coated ferrite particles showed strong magnetization and the particles could be easily attracted by magnetic field gradient caused by using magnet and so on.

### EXAMPLE 2

Polymer coated ferrite particles were obtained by using the process described in the Example 1, except replacing nonionic surface activating agent by 0.3 g of Triton X-405 given by [Chemical equation 2] having PEO chain, replacing the ionic surface activating agent by 10 μl of 10-undecenoic acid dissolved in 56 μl of 1 M NaOH solution, and replacing the water soluble initiator by 25 mg of KPS.

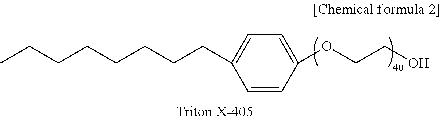

As a result of observing the obtained polymer coated ferrite particles using the transmission electron microscope, it was found that the particles were monodispersed particles carrying 1-3 ferrite particles inside each particle and having average particle diameter of 125 nm.

### EXAMPLE 3

Polymer coated ferrite particles were obtained using the process condition described in the Example 1, except changing the quantity of V-50 addition to 100 mg. As a result of observing the obtained polymer coated ferrite particles using the transmission electron microscope, it was found that the particles were monodispersed particles carrying 1-5 ferrite particles inside each particle having average particle diameter of 153 nm with the diameter standard deviation of 22 nm.

### EXAMPLE 4

Polymer coated ferrite particles were obtained using the process condition described in the Example 3, except changing the quantity of nonionic surface activating agent Emulgen 1150S-70 addition to 0.5 g. As a result of observing the obtained polymer coated ferrite particles using the transmission electron microscope, it was found that the particles were monodispersed particles carrying 1-4 ferrite particles inside each particle having average particle diameter of 90 nm with the diameter standard deviation of 22 nm.

### EXAMPLE 5

Polymer coated ferrite particles were obtained using the same composition and process condition described in the Example 1, except replacing the ferrite particles by ferrite particles having average diameter of 70 nm. As a result of transmission electron microscope observation for the obtained polymer coated ferrite particles, it was found that the particles were monodispersed particles carrying 1-3 ferrite particles inside each particle having average particle diameter of 200 nm with the diameter standard deviation of 30 nm.

### EXAMPLE 6

Polymer coated ferrite particles were obtained by using the same composition and process condition described in the Example 1, except using processes of obtaining emulsion liquid having water as continuous phase of: obtaining ferrite particle water dispersion by dispersing ferrite particles having hydrophobic character using 0.2 g of nonionic surface activating agent Emulgen 1150S-70; obtaining emulsion liquid mixing the monomer mix containing monomer, organic solvent, initiator, and cross linker agent dissolving 10 aminoundecane as surface activating agent dissolved in 56 μl of 1 M HCl solution and adding 0.1 g of nonionic surface activating agent Emulgen 1150S-70 applying ultrasonic vibration of sonication; and mixing the ferrite particle water dispersion and the emulsion liquid.

As a result of transmission electron microscope observation for the obtained polymer coated ferrite particles, it was found that the particles were monodispersed particles carrying 1-5 ferrite particles inside each particle having average particle diameter of 175 nm with the diameter standard deviation of 12 nm.

### EXAMPLE 7

Polymer coated ferrite particles were obtained using the same composition and process condition described in the Example 1, except using processes of forming emulsion liquid having water as continuous phase of: obtaining ferrite particle water dispersion prepared by dispersing ferrite particles having hydrophobic character using 0.3 g of nonionic surface activating agent Emulgen 1150S-70; obtaining monomer liquid obtained by dissolving 10 μl aminoundecane dissolved in 56 μl of 1 M HCl solution to polymer mix containing monomer, organic solvent, initiator and cross linker agent, mixing with water and giving ultrasonic vibration as sonication; and mixing the ferrite particle water dispersion and the monomer liquid.

As a result of transmission electron microscope observation for the obtained polymer coated ferrite particles, it was found that the particles were monodispersed particles carrying 1-3 ferrite particles inside each particle having average particle diameter of 120 nm with the diameter standard deviation of 12 nm.

The conditions and results of the Examples 1-7 are shown in Table 1 and Table 2.

TABLE 1

| | | Example 1 | Example 2 | Example 3 |
|---|---|---|---|---|
| Ferrite | Average diameter | 40 | 40 | 40 |
| particles | (nm) | | | |
| | Standard deviation | 10 | 10 | 10 |
| | (nm) | | | |
| Hydrophobizing agent | | 10-undecenoic acid | 10-undecenoic acid | 10-undecenoic acid |
| Surface activating agent (SA) | | | | |
| Nonionic | | Emulgen 1150S-70 | Triton X-405 | Emulgen 1150S-70 |
| | | (KAO Co.) 0.3 g | (Sigma Co.) 0.3 g | (KAO Co.) 0.3 g |
| Ionic | | Aminoundecane | 10-undecenoic acid | Aminoundecane |
| | | 10 μl 1M HCl 56 μl | 10 μl 1M NaOH 56 μl | 10 μl 1M HCl 56 μl |
| Monomer mix | | Styrene 2.7 g | Styrene 2.7 g | Styrene 2.7 g |
| | | GMA 0.3 g, | GMA 0.3 g, | GMA 0.3 g, |
| | | Diethylether 2.5 g | Diethylether 2.5 g | Diethylether 2.5 g |
| | | AIBN 0.025 g | Cross linking agent | Cross linking agent |
| | | Cross linking agent | Divinylbenzene | Divinylbenzene |
| | | Divinylbenzene | 0.08 g | 0.08 g |
| | | 0.08 g | | |
| Emulsifying process | | 1st hydrophlic -> | 1st hydrophlic -> | 1st hydrophlic -> |
| | | 2nd hydrophilic -> | 2nd hydrophilic -> | 2nd hydrophilic -> |
| | | -> Mix. monomer | -> Mix. monomer | -> Mix. monomer |
| Hydrophilic initiator | | V50 50 mg/5 ml | KPS 25 mg | V50 100 mg |
| Polymer | Average diameter | 163 | 125 | 153 |
| coated | (nm) | | | |
| ferrite | Standard deviation | 20 | 15 | 22 |
| particles | (nm) | | | |
| | Number of particles | 1-3 | 1-3 | 1-5 |
| | Magnetic property | Magnetic field | Magnetic field | Magnetic field |
| | | response: good | response: good | response: good |

TABLE 2

| | | Example 4 | Example 5 | Example 6 | Example 7 |
|---|---|---|---|---|---|
| Ferrite | Average | 40 | 70 | 40 | 40 |
| Particles | diameter (nm) | | | | |
| | Standard | 10 | 20 | 15 | 15 |
| | deviation (nm) | | | | |
| Hydrophobizing agent | | 10-undecenoic acid | 10-undecenoic acid | 10-undecenoic acid | 10-undecenoic acid |
| Surf. Activ. Agent. (SA) | | | | | |
| Nonionic | | Emulgen 1150S-70 | Emulgen 1150S-70 | Emulgen 1150S-70 | Emulgen 1150S-70 |
| | | (KAO Co.) 0.5 g | (KAO Co.) 0.3 g | (KAO Co.) 0.3 g | (KAO Co.) 0.3 g |
| Ionic | | Aminoundecane | 10-undecenoic acid | Aminoundecane | Aminoundecane |
| | | 10 μl 1M HCl 56 μl | 10 μl 1M HCl 56 μl | 10 μl 1M HCl 56 μl | 10 μl 1M HCl 56 μl |
| Monomer mix | | Styrene 2.7 g | Styrene 2.7 g | Styrene 2.7 g | Styrene 2.7 g |
| | | GMA 0.3 g, | GMA 0.3 g, | GMA 0.3 g, | GMA 0.3 g, |
| | | Diethylether 2.5 g | Diethylether 2.5 g | Diethylether 2.5 g | Diethylether 2.5 g |
| | | AIBN 0.025 g | Cross linking agent | AIBN 0.025 g | AIBN 0.025 g |
| | | Cross linking agent | Divinylbenzene | Cross linking agent | Cross linking agent |
| | | Divinylbenzene | 0.08 g | Divinylbenzene | Divinylbenzene |
| | | 0.08 g | | 0.08 g | 0.08 g |
| Emulsifying process | | 1st hydrophlic | 1st hydrophlic | Mixing: | Mixing: |
| | | -> 2nd hydrophilic | -> 2nd hydrophilic | Ferrite dispersion. | Ferrite dispersion. |
| | | -> | -> | with nonionic SA | with nonionic SA |
| | | -> Mix. monomer | -> Mix. monomer | and monomer with | and monomer with |
| | | | | nonionic and ionic | ionic SA |
| | | | | SA | |
| Hydrophilic initiator | | V50 100 mg | V50 50 mg/5 ml | V50 50 mg/5 ml | V50 50 mg/5 ml |
| Polymer | Average | 90 | 200 | 175 | 120 |
| coated | diameter (nm) | | | | |
| Ferrite | Standard | 22 | 30 | 12 | 10 |
| particles | deviation (nm) | | | | |
| | Number of | 1-4 | 1-3 | 1-5 | 1-3 |
| | particles | | | | |
| | Mag. property | Mag. field | Mag. field | Mag. field | Mag. field |
| | | response: good | response: good | response: good | response: good |

### EXAMPLE 8

Organic solvent durability test was performed for the polymer coated ferrite particles of the Example 1. Particles are examined after the test condition of immersing in solvent for 12 hours. As the result, the polymer coatings after immersion were maintained for organic solvents of methanol, ethanol, isopropanol, tetrahydrofuran, acetonitrile, acetic ether, dioxane, N, N-dimethylformamide, dimethylsulfoxide, acetone, diethylether, toluene, dichloromethane, chloroform and hexane, and durability to these organic solvents was proved.

### EXAMPLE 9

Modification of bonding EGDEs as spacers for the polymer coated ferrite particles manufactured in the Example 1 was performed.

First of all, epoxy groups of GMAs were reacted adding NH₄OH solution and adjusting pH using HCl solution. Then the epoxy groups of GMAs were opened.

Excess EGDE molecules were supplied to the amino groups appeared as a result of opening to the polymer coated ferrite particles and stirred adjusting pH with NaOH water solution, and combined a epoxy group of a EGDE molecule to a amino group of the polymer coated ferrite particles. Supplying EGDE molecules in excess, bonding formation of epoxy groups at both sides of an EGDE molecule with amino groups of polymer coated ferrite particles was avoided. After the bonding reaction, the particles were rinsed with water applying handling technique of magnetic separation. Then modified polymer coated ferrite particles having EGDE spacers were obtained.

Organic solvent durability test was performed to the polymer coated ferrite particles having EGDE modification. The test condition is the same as explained in the Example 7 of immersing for 12 hours in each solvent. As the result, the polymer coatings after immersion were maintained for organic solvents of methanol, dioxane, DMF (dimethylformamide) and DMSO (dimethylsulfoxide), and durability of the particles to these organic solvents was proved.

Furthermore, organic solvent durability tests of the EGDE modified polymer coated ferrite particles were conducted for solvents of diethylether, acetone, toluene, chloroform and hexane respectively under the same condition as explained above. As the result, the polymer coatings after immersion were maintained for each of these organic solvents, and durability to these organic solvents was also proved.

### COMPARATIVE EXAMPLE 1

Preparation of Polymer Coated Ferrite Particles were tried using the process described in the Example 6, except using only 0.3 g of water solution of nonionic surface activating agent Emulgen 1150S-70 (produced by KAO Co.), and not using ionic surface activating agent. However, the obtained polymer coated ferrite particles were polydisperse particles lacking homogeneity. It was shown that this condition of Comparable example 1 is insufficient to form homogeneous monodisperse polymer coated ferrite particles.

### COMPARATIVE EXAMPLE 2

Preparation of Polymer Coated Ferrite Particles were tried using the process described in the Example 6, except adding only 20 ml (1%) of ionic surface activating agent dodecyl sodium sulfate and not using nonionic surface activating agent. However, polymer coated ferrite particles were not formed. Then it was found that this condition of Comparable example 2 of using only ionic surface activating agent, and not using nonionic surface activating agent was insufficient for forming polymer coated ferrite particles.

### COMPARATIVE EXAMPLE 3

Preparation of Polymer Coated Ferrite Particles were tried using ferrite particles without providing hydrophobic character instead of the ferrite particles provided with hydrophilic character, and other composition and condition were the same as described in the Example 6. It was shown that this case of Comparative example 3 using ferrite particles without providing hydrophobic character, the condition is insufficient for realizing emulsion polymerization.

### INDUSTRIAL AVAILABILITY

According to the present invention, polymer coated ferromagnetic particles with very small diameter, having desirable polymer coating with sufficient magnetization for handling the particles using magnetic field can be obtained. Due to these features, the polymer coated ferromagnetic particles can be used for separating biological materials magnetically, for carriers bonding and carrying drugs to the polymer coated ferrite particles and for magnetic markers. Furthermore, wide variety of applications in industrial fields can be expected making use of these distinguishing properties.

1. A method of manufacturing a polymer coated ferromagnetic particle, comprising:
hydrophobizing one single hydrophilic ferromagnetic particle by absorbing an aliphatic acid having a hydrophobic aliphatic group and a hydrophilic acid group onto the single ferromagnetic particle to obtain a single hydrophobic ferromagnetic particle;
emulsifying the one single hydrophobic ferromagnetic particle with a monomer liquid comprising a nonionic surface activating agent which re-hydrophilizes the one single hydrophobic ferromagnetic particle, to obtain an emulsified liquid;
adding a radical addition initiator to the emulsified liquid ; and
emulsion polymerizing the monomer by radical addition polymerization.

2. The manufacturing method according to claim 1,
wherein an average particle diameter of the one single polymer coated ferromagnetic particle is from 20 to 300 nm.

3. The manufacturing method according to claim 1,
wherein the one single ferromagnetic particle comprises a hydrophobic layer on a surface thereof.

4. The manufacturing method according to claim 3,
wherein a polymer coating is adsorbed to the hydrophobic layer of the one single ferromagnetic particle.

5. The manufacturing method according to claim 1, wherein the one single ferromagnetic particle is a ferrite particle having an average particle diameter of 30 to 100 nm.

6. The manufacturing method according to claim 1, wherein the hydrophobizing aliphatic acid comprises a functional group which co-polymerizes with the monomer and forms part of the polymer.

7. The manufacturing method according to claim 1, wherein a primary component of the one single ferromagnetic particle is at least one ferrite particle selected from the group consisting of Fe₃O₄, γ-Fe₂O₃, and an intermediate between Fe₃O₄ and γ-Fe₂O₃.

8. The manufacturing method according to claim 4, wherein the polymer coating is an organic solvent durable polymer coating which maintains its shape when the single ferromagnetic particle is immersed in an organic solvent.

9. The manufacturing method according to claim 8, wherein the monomer further comprises a cross-linking agent to increase the organic solvent durability of the polymer coating.

10. The manufacturing method according to claim 9, wherein the one single polymer coated ferromagnetic particle is durable in at least one solvent selected from the group consisting of methanol, ethanol, isopropanol, tetrahydrofuran, acetoanylyl, ethyl acetate, dioxane, N,N-dimethylformamide, dimethyl sulfoxide, diethylsulfoxide, acetone, diethylether, toluene, dicloromethane, chloroform and hexane.

11. The manufacturing method according to claim 9, wherein the polymer coating comprises a surface modification which increases the organic solvent durability.
